(19) United States
(12) Patent Application Publication
(10) Pub. No.: US 2008/0311679 A1
(43) Pub. Date: Dec. 18, 2008

(54) Biosensor Device

(75) Inventors: Jacob Marinus Jan Den Toonder, Eindhoven (NL); Hendrik Roelof Stapert, Eindhoven (NL); Richard Joseph Marinus Schroeders, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/158,536

(22) PCT Filed: Dec. 20, 2006
(86) PCT No.: PCT/IB06/54977

§ 371 Date: Jun. 20, 2008

(30) Foreign Application Priority Data
Dec. 23, 2005 (EP) 05112888.2

(51) Int. Cl. G01N 33/543

(52) U.S. Cl. 436/528; 422/69

(57) ABSTRACT

The invention relates to a biosensor device with an actuating element comprising a biorecognition element that is capable of binding target molecules from a sample of interest, wherein the actuating element comprises a polymer material, can be actuated by actuating means between a first and a second position.

### FIELD OF THE INVENTION

The invention relates to a biosensor device and to a method for measuring a target molecule of interest in a sample.

### BACKGROUND OF THE INVENTION

Biosensors, by their very nature have to be very sensitive, i.e. they have to be able to detect a very low surface coverage of target, preferably better than one molecule per square micrometer. When a sample of interest is contacted with a biosensor having a biorecognition surface that contains probes that are capable of binding the target molecules of interest, the target molecules under the right incubation circumstances will bind to the probes. Most existing biorecognition surfaces, such as those used in biosensors, are immobile. These static sensors are either flat (R. Ekins, J. Clin. Lig Assay, 1996, 19(2), 146-156, U.S. Pat. No. 5,432,099), contain protrusions (WO2005022151, US2005100947) or are porous (Benoit et al., Anal. Chem 73, 2412-2420 (2001); Kessler et al., J. Clin. Microbiol, 2004, 42(5), 2173-2185). In the latter case, the fluid to be analyzed is pumped through the substrate.

For a maximum sensitivity of a test it is important to bind within a given incubation time as many target molecules as possible to the element of the biorecognition surface. The adsorption rate of targets to a substrate depends on the ‘reactivity’ or affinity of the surface and the mass transport properties of the target molecules to the surface. Binding rate can be described by r(on)=k(on)[T][S], wherein [T] equals target concentration, [S]=surface probe concentration and k(on) equals the binding constant. The mass transport properties are determined by the diffusion rate: r(diff)=D*d[T]/dx and the convection rate r(conv)=v*delta*omega*[T] wherein D is the diffusion coefficient of T, delta is the depletion layer thickness, v is the flow rate and omega is the sensor width. The adsorption rate becomes diffusion limited when the binding rate is higher than the rate of mass transport, i.e. when the depletion layer is not replenished fast enough and the probes cannot make sufficient connections with the target molecules in the sample to be measured. When the surface affinity to the target is high (k(on) is large), or when a low concentration of the target is present in the sample of interest (as is typically the case with biological or clinical samples), the rate at which the target molecules bind to the surface will become diffusion limited. However, a biosensor reaches maximum performance and speed when the adsorption kinetics is reaction limited.

Such a device is known inter alia from U.S. Pat. No. 4,956,149 wherein a biosensor is described that comprises a sensor and a piezoelectric actuator attached to one another. The sensor is a glucose sensor formed by a glucose oxidase that is immobilised in a membrane and placed on the gate electrode of a field effect transistor. The sensor is immersed in the sample to measure the possible substance to be detected (in this case glucose). By actuating the piezoelectric element, the sample is agitated and signal output is increased compared to non-agitated samples. However, in this device, the sensor and the actuating element are two distinct elements. When operated, the piezoelectric actuator is not moving in the fluid, but only the fluid is moving containing the analyte(s) and/or detection bodies. This is a different approach and a technical disadvantage in a field wherein miniaturization is progressing at high speed and whereby it is preferred that entire components can be integrated into micro fluidic systems such as described for instance in U.S. Pat. No. 6,068,751.

### SUMMARY OF THE INVENTION

It is an object of the invention to provide improved biosensors wherein the adsorption kinetics of the sensor are considerably increased and preferably beyond the diffusion-limit. It is a further object of the invention to provide for an actuator that contains integrated biorecognition elements that are capable of actually capturing the target molecules to be detected. The present inventors have found that this can be achieved when biosensors are used that contain (polymer) actuating elements combined with biorecognition elements. Thus the actuating elements of the sensor contain (an array of) biorecognition elements that can bind target molecules from a sample of interest.

In a first aspect the invention relates to a biosensor device with an actuating element comprising a biorecognition element that is capable of binding target molecules from a sample of interest, wherein the actuating element, which comprises a polymer material, can be actuated by actuating means between a first and a second position.

In a further aspect the invention relates to a method of detecting target molecules in a sample of interest comprising using the biosensor according to the invention.

### BRIEF DESCRIPTION OF THE DRAWINGS

These and other aspects of the invention are apparent from and will be elucidated with reference to the embodiments described hereinafter.

In the drawings:

FIG. 1 is a schematic side view of a biosensor according to the present invention. FIG. 1(a) is a stylized view of the sensor in a resting position, which under normal operation, is likely the starting position and the position wherein the detection of the captured target molecules takes place. FIG. 1(b) is a stylized view of the sensor in a actuated position perpendicular to the flow of the sample fluid and actively capturing target molecules. It is observed that detection can still take place in this situation. For example optical detection when focal depth of the set up is bigger than the net displacement of the actuating element.

### DETAILED DESCRIPTION OF THE PREFERRED EMBODIMENTS

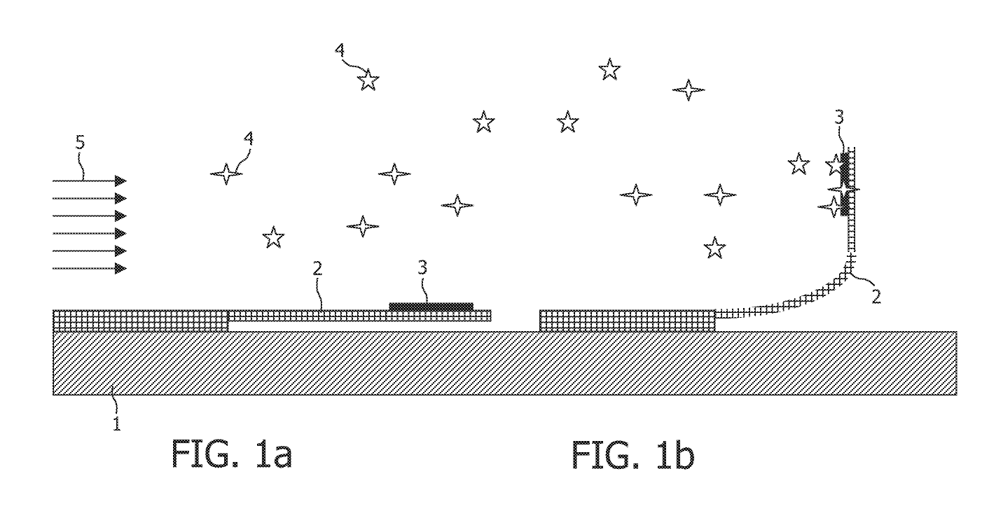

FIG. 1 shows a schematic side view of a biosensor according to the present invention. FIG. 1(a) discloses an embodiment of a biosensor (1) having an actuating (polymer) sensor element (2) in a first (non-actuated) position having a biorecognition surface element (3). When in contact with a flow (5) of a sample comprising a variety of target molecules (4), the actuating element 2 can be moved in to a second (actuated) position (b) from a first position (a), thereby obtaining improved sensitivity of the sensor through improved adsorption kinetics, i.e. reaction limited instead of diffusion limited and by obtaining better washing efficiencies.

The actuating element can consist of a flap or beam of a flexible material with a biorecognition surface attached thereto. The material is preferably a flexible polymer. In rest, the flap is in a horizontal position. The flap can be set in motion by a certain stimulus such as temperature, light, electrical field or (electro) magnetic field. Preferably a magnetic field most preferred an electro-magnetic field is used. When the sensor is actuated, the flap is moved in to a second position to come into improved contact with the fluid sample and the surface can probe into the fluid for the target molecules. By moving the flap into the second position, the area that comes into active contact with the fluid sample increases substantially as diffusion lengths to the surface decrease and the adsorption kinetics are enhanced by the actuating movement. The movement can be repeated within one measurement to further improve contact with the sample fluid.

Thus in a first aspect of the invention, there is provided a biosensor device with an actuating element comprising a biorecognition element that is capable of binding target molecules from a sample of interest, wherein the actuating element can be actuated by actuating means between a first and a second position. The activating element can preferably be reversibly attached. In certain embodiments, the actuation element comprises and preferably consists of a polymer actuating element. Such materials are known in the art, such as rubbers and elastomers (e.g. polysiloxanes); thermoplastic elastomers (such as polyesters and polyether esters or poly ether urethanes), crosslinked polymers and (hydro)gels, photopolymers, such as poly (meth)acrylates with or without mesoscopically ordered elements. Other examples are composite material structures consisting of a combination of stacked conductive and polymer films, and polymer materials with dispersed magnetic (nano-)particles (such as Fe3O4 nanoparticles). (See for example: D. J. Broer et al. Smart Materials. Chapter 4 in True Visions: Tales on the Realization of Ambient Intelligence, ed. by Emile Aarts and José Encarnaçao, Springer Verlag, 2005, or G. N. Mol et al. Adv. Funct. Mat._{—}15, 1155-1159 (2005)). Polymer actuating microelements are also described in Applicant's co-pending patent application Ser. No. “Microfluidic system based on actuator elements”. These materials are known to be able to deform significantly under a specific stimulus and hence may serve as the preferred actuating polymer materials. By using polymer-actuating elements, larger shape changes can be achieved, which allows for a more flexible design. The integration of the biorecognition element in the actuating element obviates the need for external actuators such as piezo-elements.

The surface layer of the biorecognition element can be a monolayer or multilayer with a binding agent (also referred to as probe) disposed thereon. The biorecognition element may also comprise microbeads attached to the actuating element, for instance by grafting the beads to the actuating element. The beads are preferably polymer beads. Alternatively, the biorecognition element may be in the form of a sheet or plate attached to the actuating element, which is spotted with an array of dots of binding agent (or probes). The probes may be designed to bind the target molecule reversibly or irreversibly.

In one preferred embodiment the biorecognition element is porous. In certain embodiments, the probes are located in the pores of the porous element. Use of a porous biorecognition element may cause the probe, depending on its molecular size, to be carried down into the pores of the support where its exposure to the target molecule whose concentration is to be determined may likewise be affected by the geometry of the pores. When the actuating element is actuated into the second position, which, in general, is substantially perpendicular to the direction of the flow of the sample, the fluid will flow through the porous element. The target molecules to be detected can easily be captured in the pores of the porous element.

The surface or the pores may be equipped with probes for the specific capture of target molecules. Such probes may be provided on the surface or in the pores by methods known inter alia from biotechnological microarrays (DNA-chips) such as for example those from Affymetrix, Agilent, GE healthcare, Metrigenix, Pamgene, Dow Corning, etc. Generally such probes may be selected from the group comprising an antigen, a hapten, an antibody, an antibody fragment, a DNA, PNA, LNA or RNA sequence or a combination thereof, chemicals that can react with a cell receptor, an enzyme, a protein, a (oligo- or poly)peptide, a chelate, an aptamer, a nanobody. Alternatively, depending on the actuating element material, one can choose or synthesize materials that have a high binding capacity for probes. For example, one may use a positively charged material that is able to bind DNA probes. Alternatively one may use materials that have specific reactive groups (such as carboxylic acids, epoxides, amines, sulfhydryls, etc.) that can be used to chemically anchor probe molecules to the substrate. Again alternatively, one may apply a coating on the actuating material that has high binding capacity for probes, thereby combining for example the property of actuation (bulk material) with specific surface properties (the coating). These materials, their chemistry and their synthesis methods are widely known in the art for this purpose and the skilled man can select amongst them without difficulty.

The probes used may be probes of different specificity, that is to say probes which are specific to different target molecules, or two or more of them may be probes of the same specificity but of different affinity, that is to say probes which are specific to the same target molecule but have different equilibrium constants K for reaction with it. The latter alternative is particularly useful where the concentration of analyte to be assayed in the unknown sample can vary over considerable ranges, for example 2 or 3 orders of magnitude, as in the case of HCG measurement in urine of pregnant women, where it can vary from 0.1 to 100 or more IU/ml.

The probes used are preferably antibodies or a functional part thereof, more preferably monoclonal antibodies, or functional parts thereof. Monoclonal antibodies to a wide variety of ingredients of biological fluids are commercially available or may be made by known techniques. The antibodies used may display conventional affinity constants, for example from 10exp8 or 10exp9 liters/mole upwards, e.g. of the order of 10exp10 or 10exp11 liters/mole, but high affinity antibodies with affinity constants of 10exp12-10exp13 liters/mole can also be used. The invention may be used with such probes which are not themselves labelled, but is not limited thereto.

The probes may be applied to the biorecognition element in any of the ways known or conventionally used for coating probes onto supports such as tubes, for example by contacting each spaced apart location on the support with a solution of the binding agent in the form of a small drop, e.g. 0.5 microliter, on a 1 mm² spot, and allowing them to remain in contact for a period of time before washing the drops away. Other bioprinting or embossing techniques can be likewise applied. Smaller drop volumes can be applied when using for example ink jet printing. Droplet volumes of a few picoliters to nanoliters are state of the art and result in spot diameters typically in the range of 10-500 μm, thus allowing for a large number of (different) capture spot on a small (actuating) substrate.

The target molecules are preferably selected from the group comprising DNA, RNA, proteins, peptides, antibodies, cells, electrolytes, enzymes, pharmaceutically active compounds, organic molecules or metabolites thereof. Based on the selection of the desired target molecule to be detected, the probe for its detection can be designed. For instance for the detection of the presence, absence or quantity of a compound, such as an antibody in a bodily fluid, the design of the probe is based on the antigen, or a fragment thereof that binds to the antibody. In the case of the detection of a specific DNA sequence, for instance in the case of a genetic disorder, the complementary nucleotide sequence can be designed in the form of a probe and used in the device of the present invention.

The invention may be used for the assaying of target molecules present in biological fluids, for example human body fluids such as blood, serum, saliva or urine. They can also be derived from animal, plant, or food origin or from sewage, biological waste, washing fluid etc. They may be used for the assaying of a wide variety of hormones, proteins, enzymes or other analytes which are either present naturally in the liquid sample or may be present artificially such as drugs, poisons, heavy metals or the like, The samples may be pretreated prior to the contact with the biosensor, i.e. certain assays, such as immunological assays or PCR assays may have been performed on the sample or the sample may have been subjected to an isolation or purification prior to the contact with the biosensor. In this embodiment the biosensor serves as a detection platform for a given assay.

For example, the invention may be used to provide a device for quantitatively assaying a variety of hormones relating to pregnancy and reproduction, such as FSH, LH, HCG, prolactin and steroid hormones (e.g. progesterone, estradiol, testosterone and androstene-dione), or hormones of the adrenal pituitary axis, such as cortisol, ACTH and aldosterone, or thyroid-related hormones, such as T4, T3, and TSH and their binding protein TBG, or viruses such as hepatitis, AIDS or herpes virus, or bacteria, such as staphylococci, streptococci, pneumococci, gonococci and enterococci, or funghi, such as Candida or tumour-related peptides such as AFP or CEA, or drugs such as those banned as illicit improvers of athletes' performance, or food contaminants. In each case the probes used will be specific for the target molecules to be assayed (as compared with others in the sample) and may be monoclonal antibodies or single stranded DNA probes therefor.

In certain embodiments the fluid can be a washing fluid, for instance derived from buffers or chaotropic reagentia, and may contain detergents. Washing can be performed more effectively when an actuable surface is used for the same reasons as for the detection of target molecules described herein above. Washing is preferably included to remove or reduce background signals that may arise from non-specifically adsorbed molecules or by interference from the fluid that contains the molecules.

The sensor may further comprise a detector that preferably relies on surface sensitive detection techniques. Examples thereof include the use of a (giant) magnetoresistive (GMR) sensor, evanescent wave detection (Surface Plasmon Resonance) or excitation, or evanescent wave excitation (waveguide fluorescence), impedimetric sensors, optical sensors based on surface binding luminescent molecules or reflective molecules (e.g. metals) or interferomeric layer structures.

In the sensor the signals representative of the occupancy of the probe in the samples of unknown concentrations of the target molecules can be calibrated by reference to dose-response curves obtained from standard samples containing known concentrations of the same target molecules. Such standard samples need not contain all the target molecules together, provided that each of the target molecules is present in some of the standard samples.

The detector is preferably positioned in the biosensor (1) or in its close proximity, depending on the characteristics of the detection technique. Read out of the signal may occur either from below or above the actuating substrate, depending on the detection technology. For instance, when using a GMR sensor, it is preferred that after the target molecules have been able to adsorb to the surface or in the pores of the biorecognition element, the actuator is returned to the first position for the detection of the adsorbed target molecules. Depending on the detection technique used, the dimensions of the actuable element or the surface thereof may vary. For instance in the case of GMR sensors, a thickness of the polymeric actuators can be preferably from about 0.2- to about 5 micrometer. In the case of evanescent wave sensors typically actuators of about 0.1-1 micron may be used, whereas with impedimetric sensors may require a thickness below 0.2 micron. When the read out and excitation is done from above, for example excitation of fluorescent molecules with a propagating wave and detecting the luminescence with the same optical path, the thickness of the polymer layer should be preferably such that it covers the focal depth of the set up. This may be designed such that the displacement caused by the actuating movement is still with in the focal depth of the optical system.

The device of the present invention finds preferably its application in microfluidics, and the presently described approach allows for the combination of lateral flow microfluidics with a flow-through substrate and surface sensitive detection.

In a preferred embodiment of the invention, the actuator comprises a plurality (also referred to as an array) of biorecognition elements wherein each biorecognition element is capable of binding, preferably independently, different target molecules and thereby identifying a plurality of different target molecules simultaneously in one sample. In an alternative embodiment, a set of actuators carrying biorecognition elements for a variety of target molecules are disposed side by side or sequentially in the flow of a sample to capture a variety of target molecules in a single run.

The invention in a further aspect relates to a method for the detection of target molecules in a sample of interest, comprising the steps of

bringing the sample of interest into contact with a biosensor essentially as defined hereinbefore;

actuating the polymer actuating element from the first to the second position;

allowing the target molecules to bind to the biorecognition element;

optionally, actuating the actuating element from the second to the first position; and

detecting the presence, absence or amount of the target molecules on the biorecognition element.

The device of the present invention preferably finds application as a biosensor in the preferred fields selected from molecular diagnostics, environmental monitoring and drug screening.

1. Biosensor device with an actuating element comprising a biorecognition element that is capable of binding target molecules from a sample of interest, wherein the actuating element, which comprises a polymer material, can be actuated by actuating means between a first and a second position.

2. Biosensor according to claim 1, wherein the biorecognition element comprises a surface capable of binding target molecules.

3. Biosensor according to claim 1, wherein the actuating element is activated by magnetic and/or electric field, light or temperature.

4. Biosensor according to claim 1, wherein the sensor further comprises a detector using a surface-sensitive detection or excitation technique.

5. Biosensor according to claim 1, wherein the detector is located vis-a-vis the actuating element such that when the actuating element is in the first position, the presence, absence or amount of the target molecule A can be measured.

6. Biosensor according to claim 1, wherein the target molecules are selected from the group comprising DNA, RNA, proteins, peptides, antibodies, electrolytes, pharmaceutically active compounds, organic molecules or metabolites thereof.

7. Biosensor according to claim 1, wherein the actuator element comprises a plurality of biorecognition elements wherein each biorecognition element is capable of binding different target molecules and thereby identifying a plurality of different target molecules simultaneously in a sample.

8. Biosensor according to claim 1, wherein the sample of interest is derived from humans such as saliva, sputum, blood, faeces, urine, animal, plant, food, sewage, biological waste, washing fluid origin.

9. Method for the detection of target molecules in a sample of interest, comprising the steps of
bringing the sample of interest into contact with a biosensor as defined in claim 1;
actuating the actuating element from the first to the second position;
allowing the target molecules to bind to the biorecognition element;
detecting the presence, absence or amount of the target molecules on the biorecognition element.
